(19) United States
(12) Patent Application Publication
(10) Pub. No.: US 2007/0071720 A1
(43) Pub. Date: Mar. 29, 2007

(54) Method of treating hepatitis B viral infection

(76) Inventors: Henricus Leonardus Antonius Janssen, SC Rotterdam (NL); Nigel Pluck, Oxford (GB); Matei Popescu, Basel (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 11/500,775

(22) Filed: Aug. 8, 2006

(30) Foreign Application Priority Data
Aug. 15, 2005 (EP) 05107473.0

(51) Int. Cl. A61K 38/21; A61K 31/7056

(52) U.S. Cl. 424/85.7; 514/43

(57) ABSTRACT

The present invention provides the use of PEG-IFN-α conjugates in association with ribavirin for the treatment of chronic hepatitis B infections. The present invention also provides a method for treating chronic hepatitis B infections in patients in need of such treating comprising administering an amount of PEG-IFN-α conjugate in association with an amount of ribavirin effective to treat chronic hepatitis B.

### PRIORITY TO RELATED APPLICATIONS

This application claims the benefit of European Application No. 05107473.0, filed Aug. 15, 2005, which is hereby incorporated by reference in its entirety.

### BACKGROUND OF THE INVENTION

The present invention relates to the field of treatment of chronic hepatitis B virus infections using an amount of a PEG-IFN-α conjugate in association with Ribavirin effective to treat chronic hepatitis B.

Interferons (IFNs) are naturally occurring proteins which have antiviral, antiproliferative and immunoregulatory activity. Four distinct classes of interferons are known to exist in humans (Pestka et al. (1987) Ann. Rev. Biochem. 56, 727-777 and Emanual & Pestka (1993) J. Biol. Chem. 268, 12565-12569). The IFNα family represents the predominant class of IFNs produced by stimulated peripheral blood leukocytes (Pestka et al., loc. cit.; Havell et al. (1975) Proc. Natl. Acad. Sci. USA 72, 2185-2187; Cavalieri et al. (1977) Proc. Natl. Acad. Sci. USA 74, 3287-3291), and lymphoblastoid and myeloblastoid cell lines (Familletti et al. (1981) Antimicrob. Agents. Chemother. 20, 5-9). The antiviral effect of IFNα is achieved not only by a direct influence on the viruses themselves, but by an activity on their target cells in the sense of a protection against the virus infection. The interferons can exert effects on cancer tumors and can influence the immune system of the body on that, for example, they activate macrophages and NK cells and intensify the expression of various immunologically significant constituents of the cell membrane. Details of the preparation of interferon-cDNA and the direct expression thereof, especially in E. coli, have been the subject of many publications. Thus, for example, the preparation of recombinant interferons is known, for example, from Nature 295 (1982), 503-508, Nature 284 (1980), 326-320, Nature 290 (1981), 20-26, Nucleic Acids Res. 8 (1980), 4057-4074, as well as from European Patents Nos. 32134, 43980 and 211148.

In chronic hepatitis B, combination of ribavirin and standard interferon alfa induces a lasting CD4+ T-cell proliferation and Th1 cytokine release, which enhances the probability of sustained HBV eradication (M A Rico, J A Quiroga, D Subira, S Castanon, J M Esteban, M Pardo, V Carreno: Hepatitis B virus-specific T-cell proliferation and cytokine secretion in chronic hepatitis B e antibody-positive patients treated with ribavirin and interferon alpha. Hepatology 2001, 33:295-300]. In a pilot study 24 HBeAg-negative chronic hepatitis B patients were treated with a combination of standard interferon (5 MU t.i.w.) and ribavirin (1000-1200 mg daily) for 12 months [T Cotonat, J A Quiroga, J M Lopez-Alcorocho, R Clouet, M Pardo, F Manzarbeitia, V Carreno: Pilot study of combination therapy with ribavirin and interferon alfa for the retreatment of chronic hepatits B e antibody-positive patients. Hepatology 2000, 31:502-506]. All patients were previous non-responders to interferon monotherapy. In this difficult-to-treat population after 12 months of follow-up virological response (HBV DNA negativity by PCR) was reached in 50% of the patients and normalization of ALT in 21% of the patients [T Cotonat, J A Quiroga, J M Lopez-Alcorocho, R Clouet, M Pardo, F Manzarbeitia, V Carreno: Pilot study of combination therapy with ribavirin and interferon alfa for the retreatment of chronic hepatits B e antibody-positive patients. Hepatology 2000, 31:502-506]. Thus, this treatment is not always effective.

It has been observed that in the case of IFNα, PEGylation increases circulating half-life and plasma residence time, reduces immunogenicity, decreases clearance and increases certain in vivo activities. See, e.g., Bailon, P. et al., Bioconjugate Chem. 2001, 12, No. 2, pp. 195-202. Clinical trials also appear to indicate that combination therapy of PEG-IFN-α conjugates and Ribavirin may be more effective than combination therapy of IFN-α and Ribavirin in the treatment of patients with hepatitis C, and it has been approved for that indication by the FDA.

### SUMMARY OF THE INVENTION

The present invention provides the use of PEG-IFN-α conjugates in association with Ribavirin for the treatment of chronic hepatitis B infections. In addition, the present invention provides a method for treating chronic hepatitis B infections in patients in need of such treating comprising administering an amount of PEG-IFN-α conjugate in association with an amount of Ribavirin effective to treat chronic hepatitis B. The present invention also provides a kit comprising a PEG-IFN-α conjugate and Ribavirin for the treatment of chronic hepatitis B infections.

### DETAILED DESCRIPTION OF THE INVENTION

The present invention provides an advantageous treatment of chronic hepatitis B by administering a combination of a pharmacologically effective amount of a PEG-IFN-α conjugate and a pharmacologically effective amount of Ribavirin. Preferably, said chronic hepatitis B is a HBeAg-negative or HBeAg-positive chronic hepatitis B. More particularly, the combination therapy of the present invention should provide an improved efficacy against a chronic hepatitis B which is a HBeAg-negative chronic hepatitis B. The patients are either previously untreated patients, previously treated with IFN-α and/or Ribavirin or any other drug but have subsequently relapsed, or nonresponders to previous treatment with IFN-α and/or Ribavirin or any other drug. Preferably, the patients are previous nonresponders to interferon monotherapy. Hepatitis B e antigen (HBeAg) is used to differentiate between HBeAg-positive chronic hepatitis, which is due to wild type HBV, and HBeAg-negative chronic hepatitis B, which is due to a naturally occurring HBV variant with mutations in the precore or/and basic core promoter regions of the genome.

HBeAG-negative chronic hepatitis has been recognized as increasing in many countries within the last decade and it represents the majority of cases in many countries. This form of Hepatitis B is generally associated with more severe liver disease with a very low rate of spontaneous disease remission and a low sustained response rate to antiviral therapy.

The term “PEG-IFN-α conjugate” as used herein includes IFN-αs derived from any natural material (e.g., leukocytes, fibroblasts, lymphocytes) or material derived therefrom (e.g. cell lines), or those prepared with recombinant DNA technology. Details of the cloning of IFNα and the direct expression thereof, especially in E. coli, have been the subject of many publications. The preparation of recombinant IFNαs is known, for example from Goeddel et al. (1980) Nature 284, 316-320 and (1981), Nature 290, 20-26, and European Patents Nos. 32134, 43980 and 211148. There are many types of IFNα such as IFNαI, IFNα2; and further their subtypes including but not limited to IFNα2A, IFNα2B, IFNα2C and IFNαII (also designated IFNαII or α-IFN). The term “IFNα” also includes consensus IFNα available from Amgen or mixtures of natural and/or recombinant IFNαs or IFNα variants disclosed in WO01/25438 or WO2004/046356 . The use of IFNα2A is preferred. The manufacture of IFNα2A is described in European Patents Nos. 43980 and 211148.

The IFNα is conjugated to a polymer such as a polyalkylene glycol (substituted or unsubstituted), for example, polyethylene glycol, to form PEG-IFN-α conjugate. Conjugation may be accomplished by means of various linkers known in the art, in particularly by linkers such as those disclosed in European Patent Applications, Publication Nos. 0510356, 593868 and 809996. The molecular weight of the polymer, which is preferably polyethylene glycol, may range from 300 to 70.000 daltons, and one or more, preferably one to three, polymers may be conjugated to the IFN-α. A preferred PEG-IFN-α conjugate has the formula: 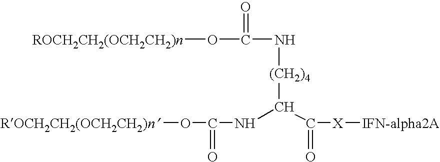
where R and R′ are methyl, X is NH, and n and n′ are individually or both either 420 or 520.

Ribavirin, 1-α-D-Ribofuranosyl-1H-1,2,4-triazole-3-carboxamide, is described in the Merck Index, compound No. 8199, Eleventh Edition. Its manufacture and formulation are described in U.S. Pat. No. 4,211,771.

The dosage of PEG-IFN-α conjugate for practicing the combination therapy of this invention is about 33 to 540 microgram (mcg) per week, more preferably 180 micrograms, in one or two weekly administrations

The dosage of Ribavirin for practicing this invention is about 400 to 1200 mg per day at least five days per week, preferably seven days per week. Based on the assumption of a patient weighing between 40 and 150 kg, the range of dosing is therefore between 10 and 30 mg per kg body weight per day. In a more specific embodiment the daily dosage of Ribavirin is 800-1200 mg, more preferably 1000 to 1200 mg. This daily dosage may be administered once per day in a single dose or in divided doses twice or thrice per day. Preferably the daily dosage of Ribavirin is administered in divided doses twice per day.

In accordance with this invention, the Ribavirin is administered to the patient in association with PEG-IFN-α conjugate, that is, the PEG-IFN-α conjugate dose is administered during the same or different periods of time that the patient receives doses of Ribavirin. In an embodiment of this invention, at least one daily dose of Ribavirin is administered within the same week as at least one dose of PEG-IFN-α. In a more specific embodiment a majority of the Ribavirin administrations occur within the same week as one or more PEG-IFN-α administrations. In another specific embodiment, all or substantially all of the Ribavirin administrations occur within the same week as one or more PEG-IFN-α administrations. At present PEG-IFN-α conjugate formulations are not effective when administered orally, so the preferred method of administering the PEG-IFN-α conjugate is parenterally, preferably by subcutaneous (sc) or intramuscular (im) injection. The Ribavirin may be administered orally in capsule or tablet form in association with the parenteral administration of PEG-IFN-α conjugate. Of course other types of administration of both medicaments, as they become available are contemplated, such as by nasal spray, transdermally, by suppository, by sustained release dosage form, etc. Any form of administration will work so long as the proper dosages are delivered without destroying the active ingredient.

The effectiveness of treatment can be determined by controlled clinical trials of the combination therapy versus monotherapy and/or combination therapy of IFN-α and Ribavirin. The efficacy of the combination therapy in alleviating the signs and symptoms of chronic hepatitis B, preferably HBeAg-negative or HBeAg-positive chronic hepatitis B infection, more preferably HBeAg-negative chronic hepatitis B infection, and the frequency and severity of the side effects will be compared with previous IFN-α monotherapy and/or combination therapy of IFN-α and Ribavirin. Three populations suffering from chronic hepatitis B infection, preferably HBeAg-negative or HBeAg-positive, more preferably HBeAg negative chronic hepatitis B, are of relevance for evaluation. Either only one or all three patient populations will be studied with the combination:
1. Patients previously untreated.
2. Patients previously treated with IFN-α and/or Ribavirin or any other drug and who had subsequently relapsed.
3. Patients who were non-responsive to previous treatment with IFN-α and/or Ribavirin or any other drug.

The effectiveness of the combination therapy will be determined by the extent to which the previously described signs and symptoms of chronic hepatitis B, preferably HBeAg-negative or HBeAg-positive, more preferably HBeAg-negative chronic hepatitis B, are alleviated.

### EXAMPLE

The primary purpose of this study is to compare the efficacy and safety of the combination of PEG-IFN-α2A and ribavirin with PEG-IFN-α2A in the treatment of HBeAg-negative chronic hepatitis B. Equal numbers of patients (61 patients per treatment group) are receiving either the combination of PEG-IFN-α2A and ribavirin or PEG-IFN-α2A for 48 weeks. A third group of patients (165 patients) is receiving PEG-IFN-α2A plus placebo for 48 weeks. The monotherapy arm provides a safety and efficacy comparator for the PEG-IFN-α2A combination arm.

The dose of PEG-IFN-α2A is 180 μg, administered sc once per week, in combination with ribavirin or placebo for 48 weeks.

The dose of ribavirin is 1000 mg or 1200 mg based upon body weight, per day in split doses. Patients weighing <75 kg (165 lbs) receive 1000 mg per day (400 mg in the morning and 600 mg in the evening), whereas patients weighing ≧75 kg receive 1200 mg per day (600 mg in the morning and 600 mg in the evening).

The primary efficacy parameters are the combined sustained virological [i.e., HBV-DNA level <10⁴ copies/ml as measured by the AMPLICOR® PCR assay (sensitivity≧100 copies/ml)] and biochemical (normalization of serum ALT concentration) responses at the conclusion of the untreated follow-up period. To be considered a responder, patients must have a normal serum alanine aminotransferase (ALT) activity at both weeks 68 and 72 and no detectable virus at week 72.

Safety assessments are performed during screening, at baseline, at weeks 1, 2, 4, 6 and 8 and then every 4 weeks thereafter throughout the 48 week treatment period. Safety assessment continues during the subsequent 24-week follow-up period. Measures of safety include adverse events, vital signs, and laboratory tests as well as tabulations of dose adjustments and premature withdrawals from treatment for safety or tolerability reasons.

Male and female patients aged 18 years or older with HBeAg-negative chronic hepatitis B who have not been treated against HBV within the previous 6 months who were not treated with any investigational drug within 30 days of entry into the present trial constitute the patient population. Patients must have quantifiable HBV-DNA, abnormal ALT on two occasions within 2 months prior to randomization (normal ALT values in between are allowed) and liver biopsy within 12 months consistent with HBeAg-negative chronic hepatitis B. Patients with other forms of liver disease, co-infection with HCV, hepatitiss D or human immunodeficiency virus (HIV), hepatocellular carcinoma, pre-existing severe depression or other psychiatric disease, cardiac disease, renal disease, seizure disorders, or severe retinopathy etc. are excluded.

A screening period (time from the first screening assessment to the first administration of test drug) of up to 4 weeks precedes treatment portion of the trial (48 weeks). Patients meeting all eligibility criteria are randomized to one of the two treatment regimens.

The primary outcome at the end of follow-up is taken as the basis for the following power analysis (appendix C). The estimated response rate is 30% for PEG-IFN monotherapy and 55% for the combination therapy. The major study question will be addressed by a pair-wise comparison between the response rates at end of follow-up. The pair-wise significance level will be set at a=0.05 (two sided testing). Power is set to 80%. Drop-outs will be calculated as non-responders at the end of follow up. Bringing the total number to be randomized to:

| | Response | Sample size final 1:1 |
|---|---|---|
| PEG-IFN monotherapy | 30% | 61 |
| Combination therapy | 55% | 61 |
| Total: | | 122 |

1. A method for treating chronic hepatitis B infections comprising administering an amount of PEG-IFN-α conjugate in association with an amount of Ribavirin effective to treat chronic hepatitis B.

2. The method of claim 1, wherein said chronic hepatitis B infection is a HBeAg-negative or a HBeAg-positive chronic hepatitis B infection.

3. The method of claim 1, wherein said chronic hepatitis B infection is a HBeAg-negative chronic hepatitis B infection.

4. The method according to claim 1, wherein the amount of PEG-IFN-α conjugate administered is about 33 to 540 mcg per week and the amount of Ribavirin administered is about 400 to about 1200 mg daily

5. The method according to claim 1, wherein the amount of PEG-IFN-α conjugate administered is 180 mcg once or twice weekly and the amount of Ribavirin administered is about 800 to about 1200 mg daily.

6. The method of claims 1 wherein the PEG-IFN-α conjugate is PEG-IFN-α2A conjugate having the formula: 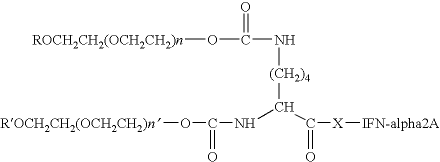
where R and R′ are methyl, X is NH, and n and n′ are individually or both either 420 or 520.

7. The method according to claim 6, wherein the amount of PEG-IFN-α conjugate administered is about 33 to 540 mcg per week and the amount of Ribavirin administered is about 400 to about 1200 mg daily

8. The method according to claim 6, wherein the amount of PEG-IFNα conjugate administered is 180 mcg once or twice weekly and the amount of Ribavirin administered is about 800 to about 1200 mg daily.

9. The method according to claim 6, wherein the amount of PEG-IFN-α conjugate administered is 180 mcg once weekly and the amount of Ribavirin administered is about 800 to about 1200 mg daily.
